(19) United States
(12) Patent Application Publication
(10) Pub. No.: US 2026/0102431 A1
(43) Pub. Date: Apr. 16, 2026

(54) Scalable Expansion of CD71+ Erythroid Progenitor Cells for Cell Therapy

(71) Applicant: University of Pittsburgh-Of the Commonwealth System of Higher Education, Pittsburgh, PA (US)

(72) Inventors: Mo Reza Ebrahimkhani, Pittsburgh, PA (US); Ryan LeGraw, Pittsburgh, PA (US)

(21) Appl. No.: 19/115,929

(22) PCT Filed: Sep. 29, 2023
(86) PCT No.: PCT/US2023/075515

§ 371 Date: Mar. 27, 2025

Related U.S. Application Data
(60) Provisional application No. 63/377,783, filed on Sep. 30, 2022.

(51) Int. Cl. A61K 35/18; A61K 40/10; A61P 37/06; C12N 5/00; C12N 5/071; C12N 5/073

(52) U.S. Cl. CPC A61K 35/18; A61K 40/10; A61P 37/06; C12N 5/0062; C12N 5/0081; C12N 5/0605; C12N 5/0672; C12N 2502/1171; C12N 2506/45; C12N 2513/00

(57) ABSTRACT

Provided herein is a scalable method of producing CD71+ erythroid progenitor cells for use in cell therapies, such as immunomodulatory therapies in which a T-cell response is suppressed. The method includes induction of a fetal liver/yolk sac organoid from pluripotent stem cells. e.g. induced pluripotent stem cells by GATA6, followed by co-culturing CD34+ cells. e.g. from cord blood or bone marrow, with the organoid, optionally in the presence of thrombopoietin, stem cell factor, and/or FLT3 ligand. Also provided herein is a bioreactor for producing the CD71+ erythroid progenitor cells.

### CROSS REFERENCE TO RELATED APPLICATIONS

This application is the United States national phase of International Application No. PCT/US23/75515, filed Sep. 29, 2023, and claims priority to U.S. Provisional Patent Application No. 63/377,783 filed Sep. 30, 2022, the disclosures of which are hereby incorporated by reference in their entireties.

### GOVERNMENT SUPPORT CLAUSE

This invention was made with government support under HL141805 awarded by the National Institutes of Health. The government has certain rights in the invention.

### REFERENCE TO A SEQUENCE LISTING

The Sequence Listing associated with this application is filed in electronic format via Patent Center and is hereby incorporated by reference into the specification in its entirety. The name of the file containing the Sequence Listing is 2305024.xml. The size of the file is 7,032 bytes, and the file was created on Feb. 27, 2025.

### BACKGROUND OF THE INVENTION

CD71+ erythroid cells (CECs) normally reside in the bone marrow and are progenitors and precursors to over 2×10¹¹ of oxygen-transporting red blood cells (RBCs) generated per day. CECs in the regulation of multiple phenomena such as fetomaternal tolerance, immune response in cancer patients, systemic inflammation in colitis, and anti-viral response in human immunodeficiency virus (HIV) infection, as well as SARS-CoV-2-induced disease (COVID-19). CD45+ CECs induced by advanced tumors inhibit CD8+ and CD4+ T cell proliferation and impair antimicrobial immunity. CECs in newborns and patients with advanced cancer have robust immunosuppressive properties. As such, the role of CECs in the regulation of immune response in both mice and humans and demonstrate that immunomodulatory properties of CECs are robust but transient and disappear during their maturation. (see, e.g., Grzywa T M, et al. Potent but transient immunosuppression of T-cells is a general feature of CD71+ erythroid cells. Commun Biol. 2021 Dec. 10; 4 (1): 1384. doi: 10.1038/s42003-021-02914-4). As such, CD71⁺ cells are expected to have significant potential in immunomodulation, and specifically in immunosuppression therapies, for example in treatment of inflammatory disease, and in transplant immunosuppression.

While showing great promise, robust and scalable methods of producing adequate quantities of CD71+ cells are lacking.

### SUMMARY OF THE INVENTION

Provided herein are scalable methods for the inexpensive production of CD71+ cells having immunosuppressive activity. Also provided herein are bioreactor devices for culturing and producing CD71+ cells having immunosuppressive activity.

According to a first aspect or embodiment, a method of preparing a population of CD71⁺ cells is provided. The method comprising: culturing pluripotent stem cells to differentiate to a fetal liver/yolk sac phenotype, such as increased expression of erythroid markers CD36, CD47, EPOR (erythropoietin receptor), and TGFβ1 (transforming growth factor β1); and coculturing CD34+ hematopoietic stem and progenitor cells (HPSCs) with the differentiated pluripotent stem cells to produce an expanded population of CD71⁺ (CD71^{HI}) cells from the HPSCs.

According to a second aspect or embodiment, a cell culture device is provided. The device comprising: a cell culture vessel (plate, flask, bioreactor, etc.) comprising cell culture media, and a coculture of cells having a fetal liver/yolk sac phenotype and CD34+ hematopoietic stem and progenitor cells.

According to a further aspect or embodiment, a method of immunosuppression in a patient in need thereof, having an inflammatory disease or an allogeneic or xenogeneic tissue transplant is provided. The method comprising administering to the patient an immunosuppressive number of CD71+ or CD71HI cells produced by the method of the first aspect or embodiment, thereby reducing a cell-mediated response in the patient.

The following numbered clauses outline various exemplary embodiments of the present invention.

Clause 1. A method of preparing a population of CD71⁺ cells, comprising: culturing pluripotent stem cells to differentiate to a fetal liver/yolk sac phenotype, such as increased expression of erythroid markers CD36, CD47, EPOR (erythropoietin receptor), and TGFβ1 (transforming growth factor β1); and co-culturing CD34⁺ hematopoietic stem and progenitor cells (HPSCs) with the differentiated pluripotent stem cells to produce an expanded population of CD71⁺ (CD71HI) cells from the HPSCs.

Clause 2. The method of clause 1, wherein the pluripotent stem cells are differentiated to a fetal liver/yolk sac phenotype by expressing a gene for expressing a GATA6 protein in the pluripotent stem cells or by introducing a GATA6 protein or mRNA encoding a GATA6 protein into the pluripotent stem cells.

Clause 3. The method of clause 2, wherein the pluripotent stem cells are differentiated to a fetal liver/yolk sac phenotype by expressing a gene for expressing a GATA6 protein in the pluripotent stem cells.

Clause 4. The method of clause 2, wherein the gene for expressing a GATA6 protein is inducible in the pluripotent stem cells.

Clause 5. The method of clause 4, wherein the gene for expressing a GATA6 protein in the pluripotent stem cells is induced during the first 3 or 4 days of culture of the pluripotent stem cells.

Clause 6. The method of clause 4 or 5, wherein the gene for expressing GATA6 is doxycycline-inducible.

Clause 7. The method of clause 2, comprising introducing a GATA6 protein or mRNA encoding a GATA6 protein into the pluripotent stem cells.

Clause 8. The method of clause 7, wherein the GATA6 protein or mRNA encoding a GATA6 protein is introduced into the pluripotent stem cells during the first 3 or 4 days of culture of the pluripotent stem cells.

Clause 9. The method of any one of clauses 1-8, wherein the pluripotent stem cells are induced pluripotent stem cells (iPSCs).

Clause 10. The method of any one of clauses 1-4, wherein the pluripotent stem cells are cultured on a basement membrane matrix such as MATRIGEL.

Clause 11. The method of any one of clauses 1-10, wherein the HPSCs are obtained from cord blood or bone marrow, such as mobilized peripheral blood.

Clause 12. The method of any one of clauses 1-11, wherein the HPSCs are obtained from a patient to whom the CD71+ cells are to be administered.

Clause 13. The method of any one of clauses 1-12, wherein the HPSCs are cocultured with the differentiated pluripotent stem cells in media comprising a factor chosen from one or more of thrombopoietin, stem cell factor, FLT3 ligand, IL-3, IL-6, and GM-CSF.

Clause 14. The method of clause 13, wherein the media comprises one or more of thrombopoietin, stem cell factor, and/or FLT3 ligand.

Clause 15. The method of clause 13 or 14, wherein the coculture media comprising the factor is at least partially changed daily.

Clause 16. The method of any one of clauses 1-15, wherein the coculturing is performed in a bioreactor, and optionally a continuous-flow bioreactor.

Clause 17. The method of any one of clauses 1-16, further comprising, after at least six days of coculturing the HPSCs with the differentiated pluripotent stem cells, separating cells of the expanded HPSCs comprising CD71+ cells from the differentiated pluripotent stem cells and collecting the separated cells of the expanded HPSCs comprising CD71+ cells.

Clause 18. The method of clause 17, further comprising enriching the separated cells of the expanded HPSCs comprising CD71+ cells for CD71+ cells by an affinity purification method.

Clause 19. The method of clause 18, wherein the affinity purification method is a fluorescence-activated cell sorting method, a magnetic bead separation method, a panning method, or a column purification method.

Clause 20. The method of any one of clauses 1-19, wherein the stem cells and/or the HPSCs are human.

Clause 21. A cell culture device comprising: a cell culture vessel (plate, flask, bioreactor, etc.) comprising cell culture media, and a coculture of cells having a fetal liver/yolk sac phenotype and CD34⁺ hematopoietic stem and progenitor cells.

Clause 22. The device of clause 20, wherein the cells having a fetal liver/yolk sac phenotype and CD34⁺ hematopoietic stem and progenitor cells are human.

Clause 23. The device of clause 20 or 21, wherein the cells having a fetal liver/yolk sac phenotype comprise an inducible gene for expressing a GATA6 protein.

Clause 24. The device of clause 23, wherein the inducible gene is doxycycline inducible.

Clause 25. The device of any one of clauses 20-23, wherein the media comprises a factor chosen from one or more of thrombopoietin, stem cell factor, FLT3 ligand, IL-3, IL-6, and GM-CSF.

Clause 26. The device of clause 24, wherein the media comprises thrombopoietin, stem cell factor, and/or FLT3 ligand.

Clause 27. The method of any one of clauses 20-25, wherein the bioreactor is a continuous-flow bioreactor.

Clause 28. The method of any one of clauses 20-26, wherein the cells of the coculture comprises, excluding the cells having a fetal liver/yolk sac phenotype, at least 10%, at least 15%, or at least 20% CD34 CD71+ cells.

Clause 29. The method of any one of clauses 20-26, wherein the cells of the coculture comprises, excluding the cells having a fetal liver/yolk sac phenotype, at least 70%, at least 75%, or at least 80% CD71+ cells.

Clause 30. A method of immunosuppression in a patient in need thereof, having an inflammatory disease or an allogeneic or xenogeneic tissue transplant, comprising administering to the patient an immunosuppressive number of CD71+ or CD71HI cells produced by the method of any one of clauses 1-19, thereby reducing a cell-mediated response in the patient.

### BRIEF DESCRIPTION OF THE DRAWINGS

FIG. 1A provides a flowchart depicting a method of producing CD71⁺ (e.g., CD17HI) cells.

FIG. 1B depicts schematically a portion of an exemplary and illustrative bioreactor device surface as described herein.

FIGS. 2A-2C provide a mRNA (cDNA) sequence (FIGS. 2A-2B, continuous, SEQ ID NO: 1), and a protein sequence (FIG. 2C, SEQ ID NO: 2) of an exemplary human GATA6 gene.

FIGS. 3A and 3B provides FACS results and graphs, respectively, depicting expansion of CD71-High Cells (CD71⁺) in FeLO/CD34⁺ coculture Relative to CD34⁺ Monoculture, as described in Example 2.

FIG. 4 provides graphs showing relative expansion of CD71+ cells in CD34⁺ Coculture is Highest in Early Stage FeLO (Day 5) relative to late stage FeLO (Day 10) and designer liver organoid (DesLO) engineered for mature liver phenotype. Y axis=CD33; X axis+CD71.

FIG. 5, provides photomicrographs of monoculture and FeLO cocultured cells stained with Wright-Giemsa stain.

FIG. 6 provides graphs showing accumulation of CD71+ cells in monoculture vs co-culture with FeLO.

FIG. 7 is a plot showing single cell RNA sequencing, indicating expression level of erythroid markers in erythroid cluster 4 generated following cultures with organoids.

FIG. 8 provides graphs showing T-cell proliferation in the presence of CD71+ cells prepared as described in the examples.

FIG. 9 Flow cytometry gating strategy and scatter plots showing increased retention of CFSE stain upon addition of CD71+ cells to culture.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values.

As used herein, “a” and “an” refer to one or more.

The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” those stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

As used herein, “treatment” or “treating” of a wound or defect means administration to a patient by any suitable dosage regimen, procedure and/or administration route of a composition, device or structure with the object of achieving a desirable clinical/medical end-point, including, for example, attracting progenitor cells, healing a wound, correcting a defect, causing neurite outgrowth, or repairing a nerve.

As used herein, the terms “cell” and “cells” refer to any types of cells from any animal, such as, without limitation, rat, mouse, monkey, and human. For example and without limitation, cells can be progenitor cells, e.g., pluripotent cells, including stem cells, induced pluripotent stem cells, multipotent cells, or differentiated cells, such as endothelial cells and smooth muscle cells. “Cells” also includes populations of cells, such as, for example, a population of cells produced by culturing CD34+ HPSCs. In certain aspects, cells for medical procedures can be obtained from the patient for autologous procedures, or from other donors for allogeneic procedures, or from xenogeneic sources.

Cell populations comprising stem cells, e.g., pluripotent and hematopoietic stem cells, may be used in the methods described herein. A stem cell may be defined as a totipotent, pluripotent, or multipotent cell of a multicellular organism from which certain other kinds of cells arise by differentiation. Stem cells are generally capable of giving rise to indefinitely more cells of the same type in cell culture. Stem cells may be characterized as totipotent, pluripotent, or multipotent, depending on their source. Stem cells may be manipulated or induced to produce GATA6 and/or other factors, e.g., by introducing a gene into the cells for expression the factors. Stem cells may be induced to differentiate, e.g. to a fetal liver/yolk sac phenotype by introduction of GATA6 or a gene for expressing GATA6, as described herein. Although obtainable from many tissue sources, non-limiting examples of tissue sources for cell populations comprising stem cells include umbilical cord stem cells (including umbilical cord blood, umbilical cord matrix, Wharton's jelly, etc. (see, e.g., Weiss M L, et al., Stem cells in the umbilical cord. Stem Cell Rev. 2006; 2(2):155-162), adipose tissue, bone marrow, perivascular cells e.g., pericytes (see, e.g., Avolio E, Alvino V V, Ghorbel M T, Campagnolo P. Perivascular cells and tissue engineering: Current applications and untapped potential. Pharmacol Ther. 2017; 171:83-92), and induced pluripotent stem cells (IPSCs, see, e.g., Yamanaka S. Induced pluripotent stem cells: past, present, and future. Cell Stem Cell. 2012 Jun. 14; 10(6):678-684 and Shi Y, et al. Induced pluripotent stem cell technology: a decade of progress. Nat Rev Drug Discov. 2017 February; 16 (2): 115-130). Cell populations useful in the present method and device may be enriched for stem cells by any useful method, including cell separation and cell sorting techniques and cell culturing techniques, as are broadly-known in the stem cell field. Methods of generation of useful iPSCs are also broadly-known. Cell populations comprising stem cells may be cryogenically preserved, e.g., in a tissue bank in which a patient's (autologous) tissue or stem cells are stored for later retrieval.

The fetal liver/yolk sac organoid (niche) may be generated from induced pluripotent stem cells (iPSCs) that are genetically engineered to contain a doxycycline-inducible GATA6 gene. Upon culture of the iPSCs in doxycyline containing pluripotency media the cells self organize and dedifferentiate into mesodermal, endodermal, and ectodermal lineages. Media is later switched to basic differentiation media. The tissue may further differentiate to achieve a gene signature aligned with a fetal liver/yolk sac phenotype, expresses an array of hematopoietic cytokines, and contains multiple cell populations derived from different lineages (endothelial, pericyte, hepatic) known to be present in the fetal liver. CD34+ hematopoietic stem and progenitor cells (HPSCs), such as cord blood derived CD34+ hematopoietic stem and progenitor cells may be seeded into a co-culture with the synthetic fetal liver/yok sac tissue. Upon co-culture the HSPCs expand in number and can be extracted at any point during the co-culture. A prominent subpopulation of the co-cultured HSPCs is a CD71+ population that expands on the fetal liver niche but is not expanded when CD34+ HSPCs are cultured in monoculture.

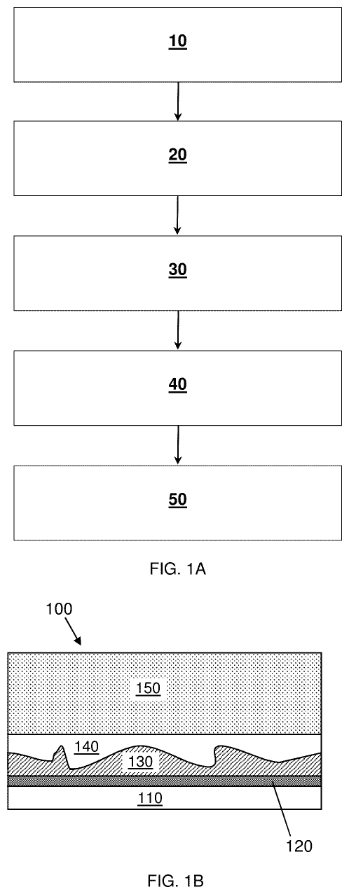

An exemplary method of producing CD71⁺ (e.g., CD17HI) CEC cells is depicted in the flowchart of FIG. 1A. In a first step 10, stem cells are cultured. The stem cells, such as human induced pluripotent stem cells (hiPSCs), may be engineered to express GATA6, e.g. human GATA6. Expression of the GATA6 may be under control of an inducible promoter, such as a doxycycline-inducible promoter (e.g., TET-ON). The stem cells may be cultured on a surface, such as a tissue culture plate, flask, or bioreactor, in stem-cell growth media until reaching a density of 20,000-40,000 cells/cm². The surface on which the cells are cultured may comprise an extracellular matrix material or basement membrane, such as an hESC-qualified growth substrate, e.g., hESC-qualified MATRIGEL. While determination of CD71^{HI} or CD71⁺ vs CD71^{LO} or CD71⁻ cells is to some extend arbitrary depending on the applicable cutoff and staining/non-staining, comparison may be made between cocultured (CD71^{HI} or CD71⁺) and monocultured (CD71^{LO} or CD71⁻) cells to differentiate the CD71^{HI} or CD71⁺ cells from CD71^{LO} or CD71⁻ cells. CD71^{HI} or CD71⁺ cells may produce a signal on staining, e.g. in a FACS or bead assay, at least 50-100× that of CD71^{LO} or CD71⁻ cells.

After the cell culture is initiated 10, expression of GATA6 is induced 20 and is maintained until fetal liver/yolk sac organoid tissue is formed. This may take from 7-14 days. Alternatively, GATA6 mRNA may be added to the cells, such as mRNA in a solid lipid nanoparticle or lipidic vesicle (see, e.g., Melamed J R, et al. Lipid nanoparticle chemistry determines how nucleoside base modifications alter mRNA delivery. J Control Release. 2022 January; 341:206-214; Hou X, et al. Lipid nanoparticles for mRNA delivery. Nat Rev Mater. 2021; 6(12): 1078-1094; and Yang L, et al. Recent Advances in Lipid Nanoparticles for Delivery of mRNA. Pharmaceutics. 2022 Dec. 1; 14(12):2682). The GATA6 may be added directly to the cells as a protein, for example in a vesicle for endocytosis, or other protein delivery methods (see, e.g., Ray M, et al. Intracellular delivery of proteins by nanocarriers. Nanomedicine (Lond). 2017 April; 12 (8): 941-952). Afterwards, CD34+ cells, such as CD34+ cord blood, bone marrow, or mobilized peripheral blood, are added to the fetal liver/yolk sac organoids and are co-cultured, e.g., with appropriate amounts of stem cell factor (SCF, e.g., human recombinant SCF), thrombopoietin (TPO, e.g., human recombinant TPO), and/or FMS-like tyrosine kinase 3 ligand (FLT3LG, e.g., human recombinant FLT3LG) effective to produce CD71+ cells. All cytokines are commercially-available and/or their amino acid and cDNA sequences are broadly-known such that a person of ordinary skill in the art can synthesize or otherwise obtain sufficient quantities of each cytokine. The amount of cytokine added is sufficient and effective to induce production of CD71+ cells from CD34+ HPSCs when co-cultured with a fetal liver/yolk sac organoid as described herein. Of note, very small quantities of the cytokines may be necessary as compared to monoculture of CD34+ HPSCs, further adding cost savings to the claimed methods, e.g., less than 500 ng/μL and at least 25 ng/μL, e.g., less than 200 ng/μL for SCF and FLT3LG, and less than 100 ng/μL for TPO, such as, for example and without limitation, rhSCF and rhFLT3LG at 100 ng/μL and rhTPO at 50 ng/μL.

A Fetal Liver/yolk sac organoid is a synthetic tissue prepared according to methods described herein. Pluripotent stem cells are cultured with GATA6 protein present, for example either expressed from a trans-gene, translated from exogenously-introduced mRNA, or introduced exogenously as a protein. The GATA6 protein, and optionally later-introduced cytokines, such as one or more of thrombopoietin, stem cell factor, FLT3 ligand, IL-3, IL-6, and GM-CSF, for example thrombopoietin, stem cell factor, and FLT3 ligand, act to differentiate the pluripotent stem cells to form an organoid that phenotypically may be identified as fetal liver or yolk sac (as a developmental precursor to fetal liver), and may have mesodermal, endodermal, and ectodermal layers, elements, or parts. Once the pluripotent stem cells are at least partially differentiated by the GATA6 protein, CD34+ HPSCs may be added, and cultured in the optional presence of the cytokines, e.g., one or more of thrombopoietin, stem cell factor, and/or FLT3 ligand for a length of time sufficient for production of CD71+ cells, such as 5, 6, 7, 8, 9, or 10 days, e.g., 6 or 7 days, at which time the CD71+ cells can be washed or aspirated from the organoids. The process may be repeated by seeding and culturing additional CD34+ HPSCs on the organoids. The pluripotent stem cells do not necessarily have to be autologous to a patient to be treated with the CD71+ cell product, but the CD34+ cells may be preferably autologous (the patient's own cells).

Hematopoietic stem and progenitor cells (HSPCs) are a cell population in the bone marrow capable of self-renewal and multi-lineage differentiation into mature blood cell types (see, e.g., Dzierzak E, Bigas A. Blood Development: Hematopoietic Stem Cell Dependence and Independence. Cell Stem Cell. 2018 May 3; 22(5):639-651). CD34+ HSPCs can be isolated from mobilized peripheral blood (See, e.g., Pelus L M, Broxmeyer H E. Peripheral blood stem cell mobilization; a look ahead. Curr Stem Cell Rep. 2018 December; 4 (4): 273-281), bone marrow, and umbilical cord blood. The HPSCs may be obtained from cord blood of the patient (if available), or from the patient's bone marrow cells, such as obtained from the patient's bone marrow, or mobilized bone marrow cells available in a patient's blood.

A “cell growth matrix” is a mesh, matrix, particle, surface, hydrogel, porous structure, or other material upon which or into which a cell can be deposited and can be maintained in a living state, and often propagates (multiplies) in the presence of suitable cell growth media. A cell growth matrix can be manufactured from a single composition, or multiple compositions, such as synthetic and/or natural polymer compositions. A cell growth matrix may comprise cells and/or therapeutic agents. A “scaffold-free cell growth matrix” contains no synthetic polymeric compositions and is a natural product of cells and tissues. In the context of the present invention and disclosure, a scaffold-free cell growth matrix may be a basement membrane secreted from cells, such as MATRIGEL.

FIG. 1B depicts schematically a portion of an exemplary bioreactor device 100 having a bioreactor substrate surface 110, having an optional basement membrane 120, such as MATRIGEL, deposited thereupon, a layer of differentiated fetal liver/yolk sac organoid cells 130, which may be a continuous or discontinuous layer depending on growth and differentiation patterns, a layer of CD34+ and/or CD71+ cells 140 over the organoid cells 130, and suitable media 150. The bioreactor may be a dish, a multi-well plate, a flask, a tube, a container, or any other suitable configuration for a bioreactor, with FIG. 1B depicting a media-facing surface of the bioreactor device. Of note, any portion of the culturing method may be automated using suitable robotic and fluidic options, as would be understood by those of ordinary skill in the art. Cells containing a population of CD71+ cells may be collected by washing, followed by filtration or centrifugation and affinity purification methods, such as, for example and without limitation by FACS or bead-based sorting (e.g., magnetic beads).

A composition is “free” of a stated constituent if that constituent is not present in the composition or is present in insubstantial amounts that do not interfere, or that insignificantly interfere, with intended use and function of the composition.

A “population of cells” are cells refers to two or more cells. The cells in a population of cells may be the same, as in an enriched, purified, or clonally expanded population of stem cells or CD71+ cells. The cells in a population of cells may comprise different cell types, as in cells obtained directly from a tissue sample, comprising stem cells as well as differentiated cells.

CD71+ cells may be used as immunosuppressive agents. As such, the CD71+ cells prepared by the method described herein may be prepared from CD34+ cells, such as bone marrow stem cells derived from the patient to be treated, such that the CD71+ cells are autologous. In one example, the patient may be in receipt of a tissue or organ transplant, such that the CD71+ cells are administered to the patient in an amount effective to suppress rejection of the transplanted tissue or organ. A person of ordinary skill can determine how many cells, and on what dosing regimen, to effectively reduce transplant rejection. Likewise diseases resulting from T cell-mediated inflammation may prove treatable with the described cells.

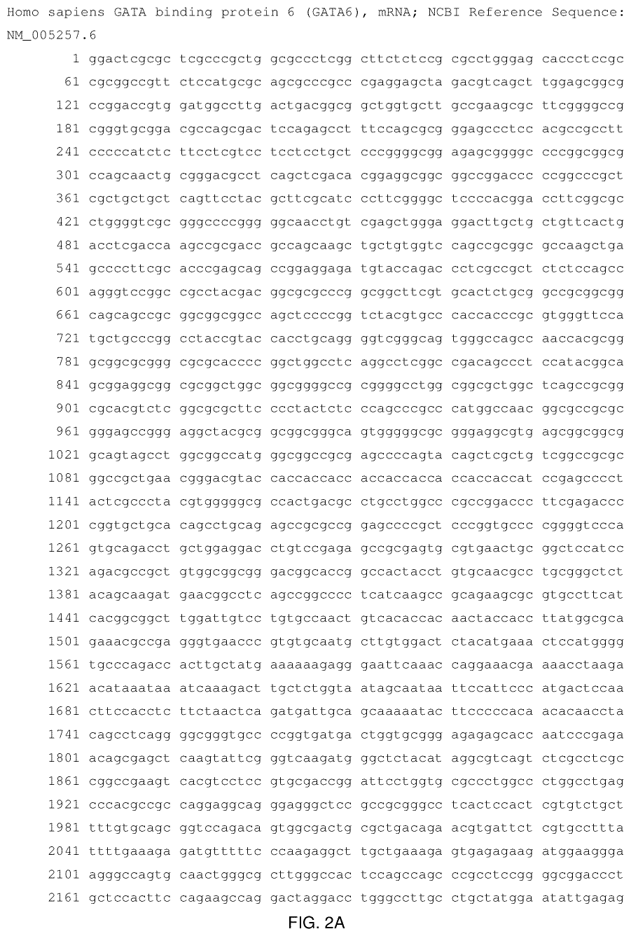

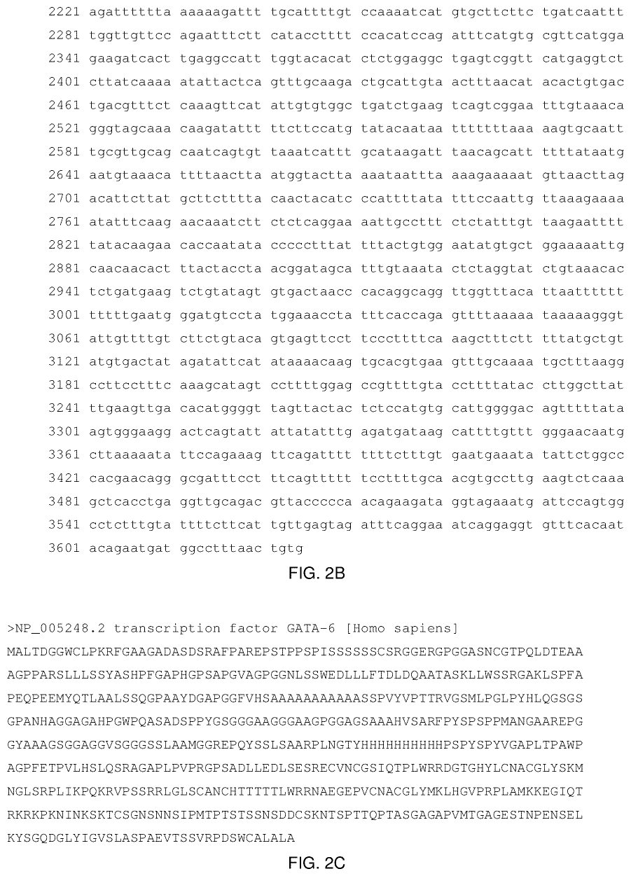

GATA6 refers to GATA binding protein 6 and is a member of the GATA family of developmentally-expressed transcriptional regulatory proteins. GATA factors constitute a family of transcriptional regulatory proteins expressed with distinct developmental and tissue-specific profiles and are thought to regulate cell-restricted programs of gene expression. Suzuki et al. (The human GATA-6 gene: structure, chromosomal location, and regulation of expression by tissue-specific and mitogen-responsive signals. Genomics. 1996 Dec. 15; 38(3):283-90) described the molecular cloning, chromosomal location, and the transcription of the human GATA6 gene. The cDNA encodes a predicted 449-amino acid protein that is highly conserved among vertebrates and includes 2 adjacent zinc finger/basic domains characteristic of the GATA factor family (e.g., mouse, rat, zebrafish, pig, sheep, Xenopus, cattle, rhesus monkey, etc.). For purposes herein although human GATA6 is described, it is expected that other vertebrate GATA6 genes, including variants and alleles, would be effective in the methods and systems described herein. FIGS. 2A-2C provide an exemplary human GATA6 cDNA and protein sequence.

By “expression” or “gene expression,” it is meant the overall flow of information from a gene. A “gene” is a functional genetic unit for producing a gene product, such as RNA or a protein in a cell, or other expression system encoded on a nucleic acid and generally comprising: a transcriptional control sequence, such as a promoter and other cis-acting elements, such as transcriptional response elements (TREs) and/or enhancers; an expressed sequence that typically encodes a protein (referred to as an open-reading frame or ORF) or functional/structural RNA; and a polyadenylation sequence). A gene produces a gene product (typically a protein, optionally post-translationally modified or a functional/structural RNA) when transcribed. By “expression of genes under transcriptional control of,” or alternately “subject to control by,” a designated sequence such as a promotor, it is meant gene expression from a gene containing the designated sequence operably linked (functionally attached, typically in cis) to the gene. A gene that is “under transcriptional control” of a promotor or transcription control element, is a gene that is transcribed at detectably different levels in the presence of a transcription factor, e.g., in the presence of a suitable chemical compound, such as doxycycline in the case of a dox-responsive promoter, such as a tet-inducible promoter. A “gene for expression of” a stated gene product, such as GATA6 is a gene capable of expressing that stated gene product when placed in a suitable environment, that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, such as the tissue specific promoters described herein, “suitable conditions” means when factors that regulate transcription, such as DNA-binding proteins, are present or absent, for example, an amount of the respective inducer is available to the expression system (e.g., cell), or factors causing suppression of a gene are unavailable or displaced-effective to cause expression of the gene.

Transcriptional control elements include promoters, enhancers, transcription factor-responsive elements (TREs, e.g., transcription factor binding sequences), suppressors, introns, etc., as are broadly-known. Additional transcription control elements, such as a WPRE (woodchuck hepatitis virus post-transcriptional regulatory element), or an intron, e.g., as shown below, which can increase expression from certain viral vectors, can be included in the gene.

A gene may be introduced into a cell, such as a pluripotent stem cell, e.g., an iPSC, by any useful method, such as by viral transduction (e.g., AAV or lentiviral transduction), PiggyBac transposon, or any other useful transformation or transduction method. A person of ordinary skill in the molecular biology art would be able to introduce a gene for inducible expression of GATA6 in a pluripotent stem cell without undue experimentation. Various cloning and transformation/transduction vehicles, such as plasmids, that GATA6 can be introduced into, are broadly available, such as from Addgene, among many other vendors.

Further to the above, CD71+ cells are circulating erythroid cells (CECs) that reside in the bone marrow, but in response to physiologic or pathologic stress conditions (e.g., anemia, cancer) may become enriched in organs outside the bone marrow. In addition to their role in production of erythrocytes, CECs have been shown to play a role in modulation of the immune system. Neonatal CECs may contribute to their vulnerability to infectious diseases but suppress activation of immune cells in response to abrupt colonization with commensal microorganisms after delivery. The peripheral blood and placenta of pregnant women are enriched in pregnant women and are responsible for the regulation of feto-maternal tolerance. Recent studies have revealed a role for CECs in HIV and SARS-CoV-2 infections.

CECs suppress the proinflammatory responses of myeloid cells and T cell proliferation by the depletion of L-arginine by arginase. CECs also produce reactive oxygen species to decrease T cell proliferation. CECs secrete cytokines, including transforming growth factor β (TGF-β), which promotes the differentiation in regulatory T cells.

HSPC expansion is of demand due to the need for therapeutic transplantation of HSPCs for hematologic diseases. Previous methods have utilized culture systems that supplement exogenous factors into the media to facilitate HSPC expansion including fetal bovine serum, low density lipoproteins, and cytokines such as thrombopoietin, stem cell factor, FLT3 ligand, IL-3, IL-6, and GM-CSF. The fetal liver niche system requires minimal addition of the aforementioned factors and thus presents a cheaper method to expand HSPCs. Also due to the adherent nature of the tissue, this system is easily scalable to large vessels, bioreactors, bead cultures, and similar systems with high surface area.

Specifically, the fetal liver niche has the unique ability to expand the HSPC subpopulation of CD71-high cells that are minimally expanded with current protocols. Due to their immunosuppressive nature, CD71+ cells have potential cell therapy applications for immune modulation and suppression of immune response, such as during organ transplant or as a therapy for autoimmune conditions. The ability to expand this population in a cheap and scalable way presents an opportunity for the bio-manufacturing of immunosuppressive cell therapies.

This system presents a scalable and cheaper alternative to previous methods used for HSPC expansion. Furthermore it has the capacity to expand cell populations (CD71+) that undergo negligible expansion using previous methods.

Unique features of the technology described herein includes:
Synthetic fetal tissue developed from single, expandable starting population
Expansion of CD71+ population that is not possible using previous methods. CECs are known to have immunosuppressive features so a novel way to expand this population presents a more financially viable way to produce a cell therapy for immunosuppression.
Tissue is growth is scalable as a function of culture surface area so it easily scalable to larger culture conditions.
Cord blood CD34+ HSPCs can be expanded with minimal addition of exogenous factors added in previous methods including fetal bovine serum, low density lipoproteins, and cytokines such as thrombopoietin, stem cell factor, FLT3 ligand, IL-3, IL-6, and GM-CSF.

Guye et al. (Genetically engineering self-organization of human pluripotent stem cells into a liver bud-like tissue using Gata6. Nat Commun. 2016 Jan. 6; 7:10243) describe production of fetal liver organoids using Dox-inducible GATA6-transduced human induced pluripotent stem cells (hiPSCs). HiPSCs were transduced with engineered lentiviral vectors containing the human GATA6 gene under control of a doxycycline-inducible promoter. While the dox (tet) promoter is very common and well-studied, other inducible promoter systems, e.g., as are known to those of skill in the genetic engineering arts, suitable for expression in stem cells, such as iPSCs, may be employed. Suitable hiPSCs are broadly-available (e.g., ATCC-HYR0103 Human Induced Pluripotent Stem (IPS) Cells, among others).

Delyea, C. et al. (Delyea, C., Elahi, S., CD71+Erythroid Suppressor Cells Promote Fetomaternal Tolerance through Arginase-2 and PDL-1. J Immunol. 2018 Jun. 15; 200(12):4044-4058) provides experimental evidence for the role and mechanism of CD71+ cells. They demonstrate that CD71+ erythroid cells are expanded at the fetomaternal interface and in the periphery during pregnancy in both humans and mice. They show that these cells exhibit immunosuppressive properties, and their abundance is associated with a Th2 skewed immune response, as their depletion results in a proinflammatory immune response at the fetomaternal interface. In addition to their function in suppressing proinflammatory responses in vitro, maternal CD71+ erythroid cells inhibit an aggressive allogeneic response directed against the fetus such as reduction in TNF-α and IFN-γ production through arginase-2 activity and PD-1/programmed death ligand-1 (PDL-1) interactions. Their depletion leads to the failure of gestation due to the immunological rejection of the fetus. Similarly, fetal liver CD71+ erythroid cells exhibit immunosuppressive activity. Therefore, immunosuppression mediated by CD71+ erythroid cells on both sides (mother/fetus) is crucial for fetomaternal tolerance. The study revealed a previously unappreciated role for CD71+ erythroid cells n pregnancy and indicate that these cells mediate homeostatic immunosuppressive/immunoregulatory responses during pregnancy. While this study provides important evidence for the immunomodulatory role of CD71+ cells, it does not provide any method for biomanufacturing of these cells. It also only focuses on the physiological role of these cells in vivo and does not explore how these cells could be leveraged for other therapies.

To assess the biological role of CD71+ cells in systemic inflammation, Kanemasa et al. (Kanemasa, H., et al. The immunoregulatory function of peripheral blood CD71+ erythroid cells in systemic-onset juvenile idiopathic arthritis. Sci Rep. 2021 Jul. 13; 11(1):14396) investigated the gene expression and function in systemic-onset juvenile idiopathic arthritis (SoJIA). Peripheral blood mononuclear cells of SoJIA patients expressed upregulated erythropoiesis-related genes. It represented the largest expansion of CECs during active phase SoJIA among other inflammatory diseases. Circulating CECs counts in inflammatory diseases were positively correlated with the levels of C-reactive protein, IL-6, IL-18, or soluble TNF receptors. Co-culture with active SoJIA-driven CECs suppressed secretions of IL-1β, IL-6, and IL-8 from healthy donor monocytes. CECs are driven to the periphery during the acute phase of SoJIA at higher levels than other inflammatory diseases. This study concluded that circulating CECs may control excessive inflammation via the immunoregulatory pathways, partly involving arginase-2. While this study highlighted the role of CD71+ cells in systemic inflammation and provided further evidence for the potential therapeutic value as a cell therapy, it only focuses on the physiological role of these cells in vivo and does not explore how these cells could efficiently biomanufactured or be leveraged for other therapies.

Previous erythroid cell cultures have depended on added serum or erythropoietin. Juutistenaho, S., et al. (Juutistenaho, S., Kekomaki, R., Growth of erythroid cells from thawed unseparated cord blood in vitro without exogenous erythropoietin. Transfus Apher Sci. 2013 October; 49 (2): 193-9) reported the growth of erythroid cells from thawed unseparated cord blood units in vitro without serum or exogenous erythropoietin. However, this was discovered in a system optimized for megakaryocytic erythroid differentiation and does not yield high numbers of erythroid cells. Furthermore the goal of this study is to generate mature erythrocytes that do not possess immunosuppressive activity.

Trakarnsanga, K., et al. (Trakarnsanga, K., Frayne, J., An immortalized adult human erythroid line facilitates sustainable and scalable generation of functional red cells Nat Commun. 2017 Mar. 14; 8:14750) created a platform for erythroid generation by immortalizing early adult erythroblasts generating a stable line to provide a continuous supply of red cells. The immortalized cells differentiate efficiently into mature, functional reticulocytes that can be isolated by filtration. Extensive characterization did not any differences between these reticulocytes and in vitro-cultured adult reticulocytes functionally or at the molecular level, and importantly no aberrant protein expression. This study demonstrates a feasible approach to the manufacture of red blood cells for clinical use from in vitro culture. However this study still has optimized protocols for mature erythrocyte generation, not for CD71+ erythroid cells with immunosuppressive function. Therefore this study represent a method for erythrocyte generation but not for the biomanufacturing of immunosuppressive CD71+ cells for cell therapy applications.

Elahi, S. et al. (Elahi, S., Oyegbami, O., CD71+Erythroid Cells in Human Neonates Exhibit Immunosuppressive Properties and Compromise Immune Response Against Systemic Infection in Neonatal Mice. Front Immunol. 2020 Nov. 24; 11:597433) demonstrates that CD71+ cells (CECs) are abundant in the peripheral blood of human newborns. Although their frequency appears to be more variable compared to their counterparts in mice, they rapidly decline by 4 weeks of age. However, their proportion remains significantly higher in infants up to six months of age compared to older infants. They found CECs from human newborns suppressed cytokine production by CD14 monocytes and T cells, which was partially abrogated by apocynin in vitro. Moreover, the depletion of CECs in neonatal mice increased the number of activated effector immune cells in their spleen and liver, which rendered them more resistant to Listeria monocytogenes infection. This study provides further evidence for the immunosuppressive effect of CECs and provides a mechanism. However it also only focuses on the physiological role of these cells in vivo and does not explore how these cells could efficiently biomanufactured or be leveraged for other therapies.

Shahbaz, S., et al. (Shahbaz, S., Elahi, S., CD71+ VISTA+ erythroid cells promote the development and function of regulatory T cells through TGF-β. PLoS Biol. 2018 Dec. 14; 16(12):e2006649) show that neonatal CD71+ erythroid cells express significant levels of V-domain Immunoglobulin (lg) Suppressor of T Cell Activation (VISTA) and, via constitutive production of transforming growth factor (TGF)-β, play a pivotal role in promotion of naïve CD4+ T cells into regulatory T cells (Tregs). They show that CD71+VISTA+ erythroid cells-compared to CD71+VISTA− and CD71+ erythroid cells from the VISTA KO mice-significantly exceed promotion of naïve CD4+ T cells into induced Tregs (iTreg) via TGF-β in vitro. They demonstrate that iTreg development by CD71+ erythroid cells is mediated through the inhibition of key signaling molecules phosphorylated protein kinase B (phospho-Akt) and phosphorylated mechanistic target of rapamycin (phospho-mTOR). They also found that elimination of Tregs using forkhead box P3 (FOXP3)-diptheria toxin receptor (DTR) mice resulted in a significant expansion in the frequency of CD71+ erythroid cells in vivo. This provides insights into the cross-talk between CD71+ erythroid cells and Tregs in newborns and highlight the biological role of CD71+ erythroid cells in the neonatal period and possibly beyond. This study further confirms the potential for CD71+ cells to be used as an immunosuppressive cell therapy.

### EXAMPLES

Generation of CD71+ Cells Via CD34+ Cord Blood Cell Coculture with Fetal Liver Organoid (FeLO)

### Materials:

GATA6-inducible engineered hiPSC
hESC qualified Matrigel (Corning Cat #354277).
Human cord blood derived CD34+ cells
Doxycycline [1 mg/mL stock] (StemCell Technologies, Cat #72742)
mTeSR1 (StemCell Technologies, Cat #85850).
IMDM 1×[L-Glutamine, 25 mM HEPES] (ThermoFisher, Cat #12440053)
   Pen Strep [10k Units/mL Penicillin, 10k ug/mL Streptomycin [100×]] (ThermoFisher, Cat #15140122)
   MEM Nonessential Amino Acids solution [100×] (ThermoFisher, Cat #11140050) (NEAA)
   GlutaMAX supplement [100×] (ThermoFisher, Cat #35050061)
   FBS (from vendor of choice)
rhFLT3L (R and D Systems, Cat #308-FK)
rhTPO (R and D Systems, Cat #288-TP)
rhSCF (R and D Systems, Cat #255-SC)
CD71 Microbeads (Miltenyi, Cat #130-046-201)

### Procedure:

1. Initiate cultures by seeding GATA6-inducible engineered hiPSC at a cell density of 30k/cm²,
2. The next day, prepare mTesR1 with doxycycline to a final concentration of 1 ug/mL (1:1000),
3. Replace media with 1 μg/mL Dox-mTeSR1 media,
4. Exchange media daily until day 5,
5. On day five, replace Dox-mTeSR1 media with IMDM optionally supplemented with 10% FBS, 1× GlutaMAX, 1× Pen Strep, and 1×NEAA,
6. Exchange daily until day 10,
7. On day 10 seed CD34+ cord blood cells at 10k/cm² using IMDM optionally supplemented with 10% FBS, 1× GlutaMAX, 1× Pen Strep, and 1×NEAA, rhSCF and rhFLT3LG at 100 ng/ul and rhTPO at 50 ng/ul,
8. Manually exchange up to the whole volume of media per well (media described in step 7) daily or every other day by aspiration with a micropipette,
9. After up to 20 days of coculture collect the expanded CD34+ cord blood cells from the FeLO by aspirating the media, washing once with PBS, and combining the PBS wash volume with the initial aspirate,
10. Isolate CD71+ population by magnetic based MACS separation using CD71 microbeads (according to manufacturer's protocol) or flow cytometry sorting for CD71.
   Monoculture Vs. Co-Culture

CD71+ cells were prepared essentially according to the coculture method provided above. For preparation of monocultures, CD34+ cells were isolated from umbilical cord blood according to the manufacturer's protocol for the EasySep Human Cord Blood CD34 Positive Selection Kit II (Stem Cell Technologies Cat #17896). Fractionation of cord blood was performed by diluting the blood 1:1 with PBS containing 2% FBS and 1 mM EDTA, gently layering 30 mL diluted blood over 15 mL of Lymphoprep (Stem Cell Technologies Cat #07801), and centrifuging tubes at 1200×g for 20 minutes with brake off. The buffy coat (enriched mononuclear cell layer) was gently removed and washed with PBS containing 2% FBS and 1 mM EDTA. CD34+ selection cocktail and RapidSpheres were incubated with the mononuclear cells and CD34+ cells were magnetically isolated using “The Big Easy” EasySep magnet (Stem Cell Technologies Cat #18001) according to the manufacturer's instructions. Isolated CD34+ cells were either used immediately or cryopreserved.

Cord blood CD34+ control monocultures were cultured in IMDM as basal media supplemented with FBS at 10% and GlutaMax, MEM Non-essential Amino Acids Solution, and Penicillin-Streptomycin at 1× supplemented with 100 ng/ml human stem cell factor, 100 ng/mL Flt3 ligand, and 50 ng/mL human thrombopoietin. Cord blood CD34+ cells were seeded at 40,000 cells/mL and half the media volume per well was manually exchanged daily.

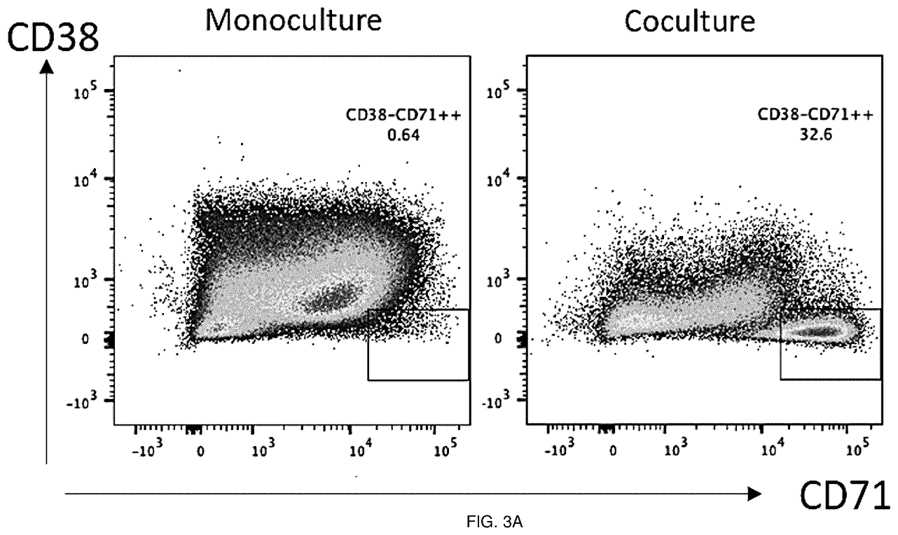

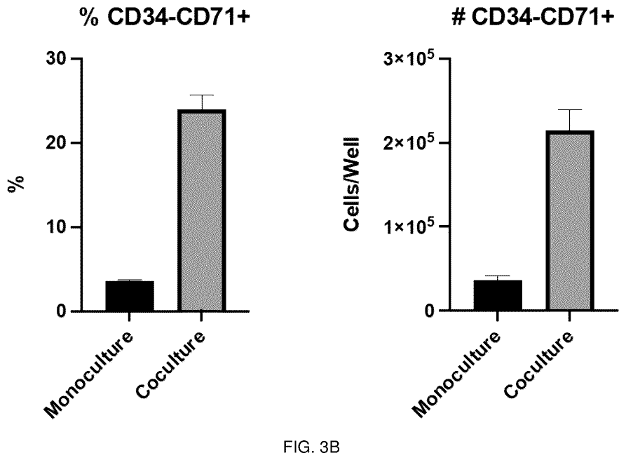

Monocultured and cocultured CD71+ cell were evaluated by FACS for CD38 and CD71 expression. As shown in FIGS. 3A and 3B, the coculture method produced significantly more CD71+ cells as compared to the monoculture method.

Monoculture Vs. Co-Culture in FeLO and Designer LO

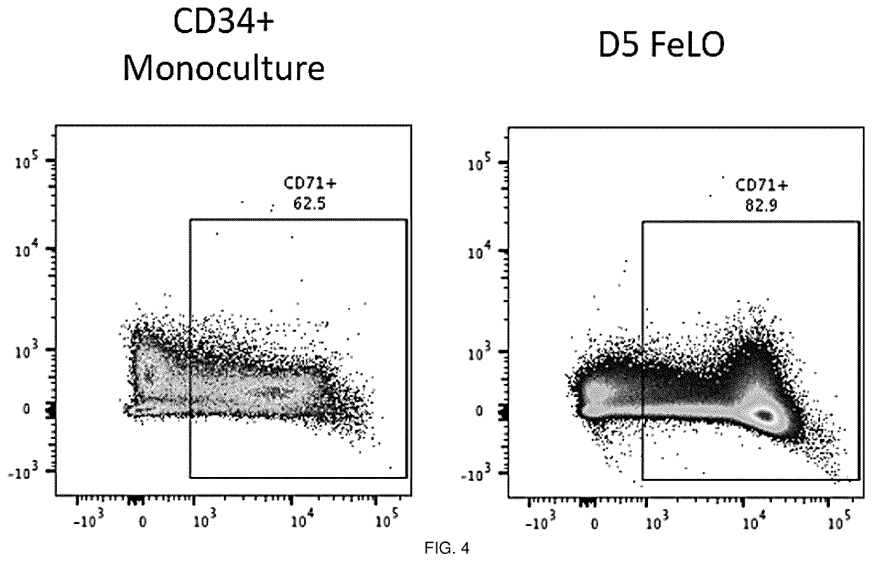

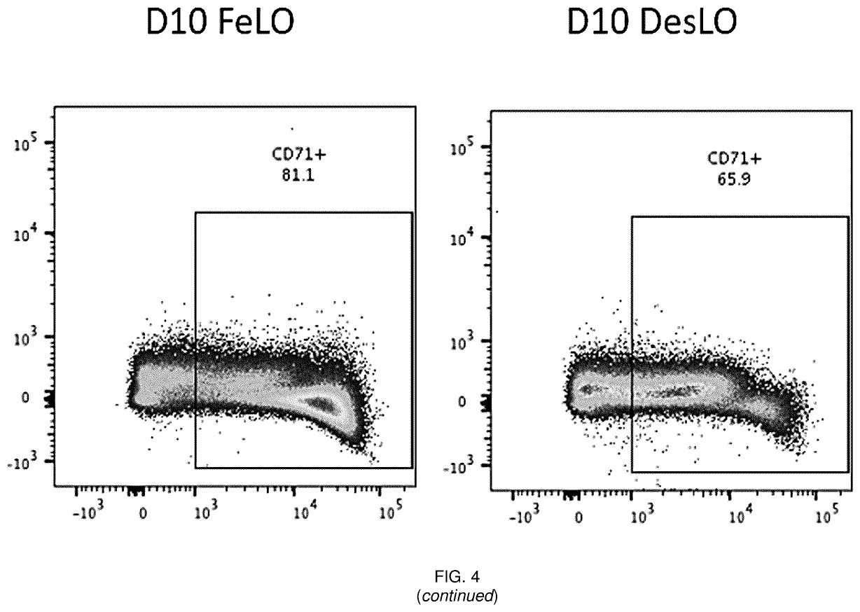

CD71+ cells were prepared essentially as described above. CD71+ cells also were expanded using a designer liver organoid (DesLO) engineered for mature liver phenotype, essentially as indicated in Velazquez J J, et al. Gene Regulatory Network Analysis and Engineering Directs Development and Vascularization of Multilineage Human Liver Organoids. Cell Syst. 2021 Jan. 20; 12(1):41-55.e11. doi: 10.1016/j.cels.2020.11.002. Epub 2020 Dec. 7. As shown in FIG. 4, the FeLO coculture method is clearly superior to monoculture and coculture using the DesLO material, with slightly higher numbers of CD71+ cells at day 5 of coculture.

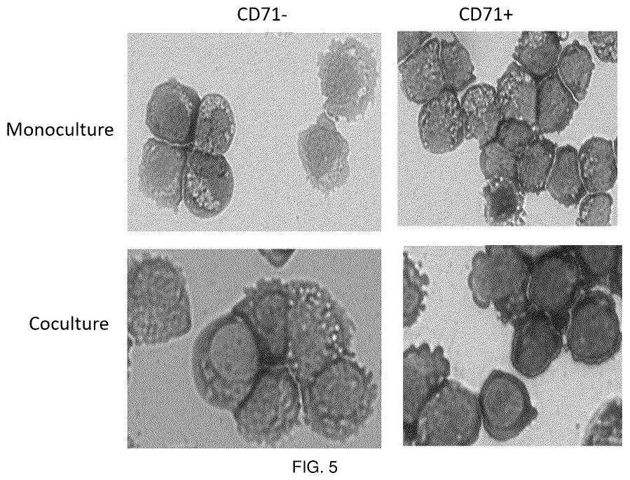

Cells produced from the monoculture and FeLO coculture were stained with Wright-Giemsa stain. As shown in FIG. 5, Coculture-expanded CD71+ cells exhibit large nucleus and lack of granules characteristic of CECs.

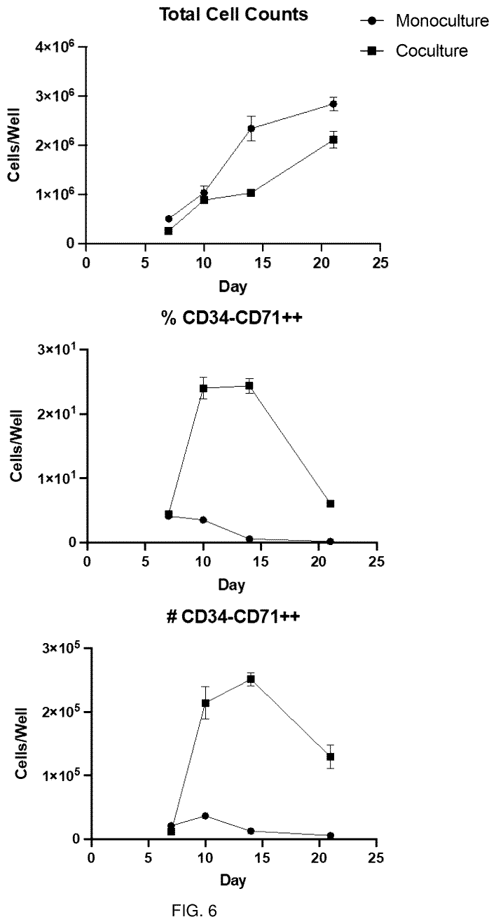

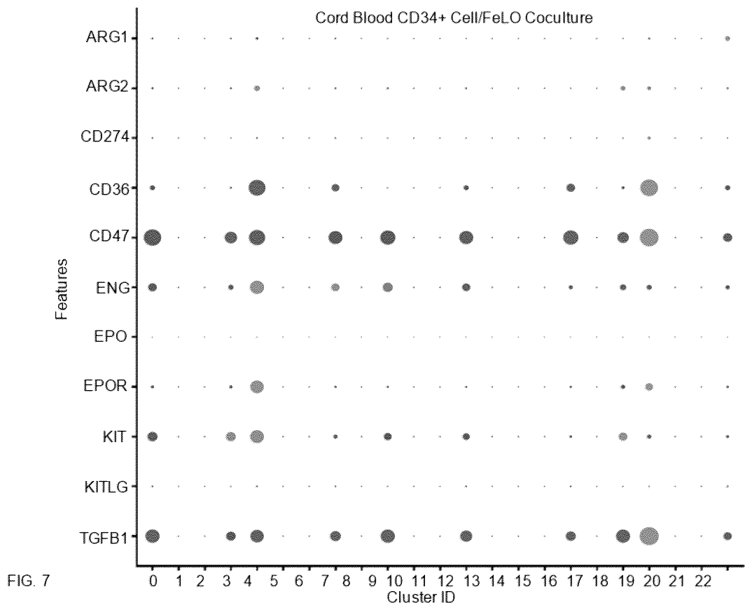

FIG. 6 depicts cell counts of CD71+ cells in monoculture as compared to co-culture, indicating significant accumulation of CD71+ cells beginning at day 7 of co-culture. Single cell RNA sequencing indicates expression level of erythroid markers in erythroid cluster 4 in co-cultured cord blood CD34⁺ cells as shown in FIG. 7.

An in vitro Mixed Lymphocyte Reaction Assay was conducted to determine if CD71+ erythroid cells generated in vitro suppress the proliferation of T cells post activation. Briefly:

### Experimental Approach: Beads Activation

1—Enrich for CD3 T cells using MojoSort Human CD3 kit;
2-Stain enriched CD3 T cells with CFSE (Proliferating cells will be CFSE−);
3-Stimulate cells with 2.5 ml of anti-CD3 anti-CD28 Beads or allogenic dendritic cells (DCs);
4—Coculture CD3 T cells along with CD71+ erythroid cells; and
5—Following 4 days of coculture, run flow cytometry.

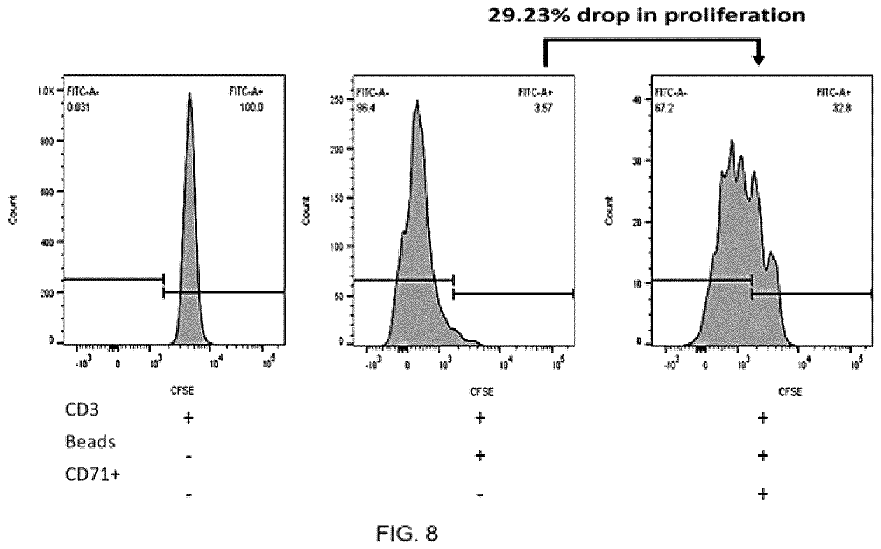

A 29.23% drop of T-cell proliferation was seen in the presence of CD71+ cells prepared using the described FeLO co-culture method (FIG. 8).

For Flow cytometry analysis, human CD3+ T cells were cultured with dendritic cells in either the presence or absence of CD71+ cells and stained with CellTrace CFSE cell proliferation kit (Thermo Fisher Scientific Cat #C34554) according to the manufacturer's protocol. Cells were cultured for 24 hours, stained with an antibody for human CD3, and analyzed via flow cytometry for the CFSE proliferation marker.

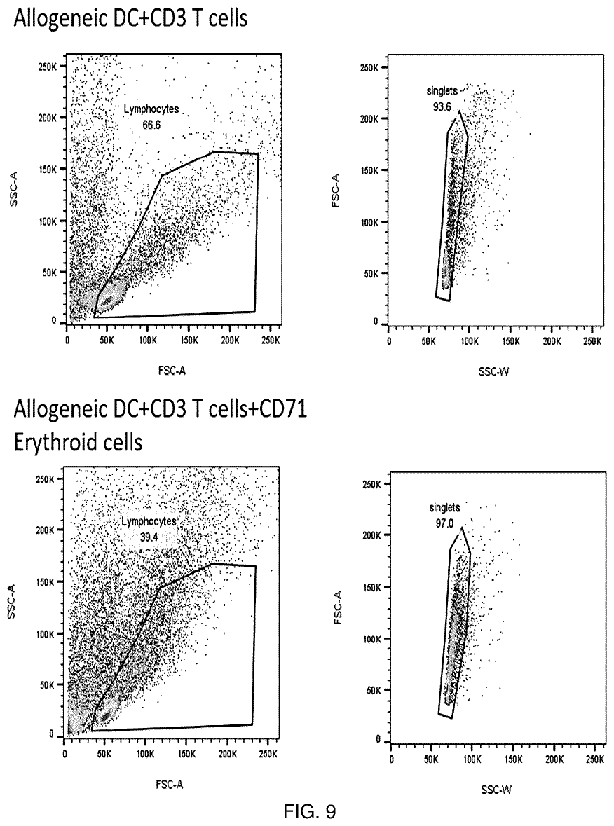

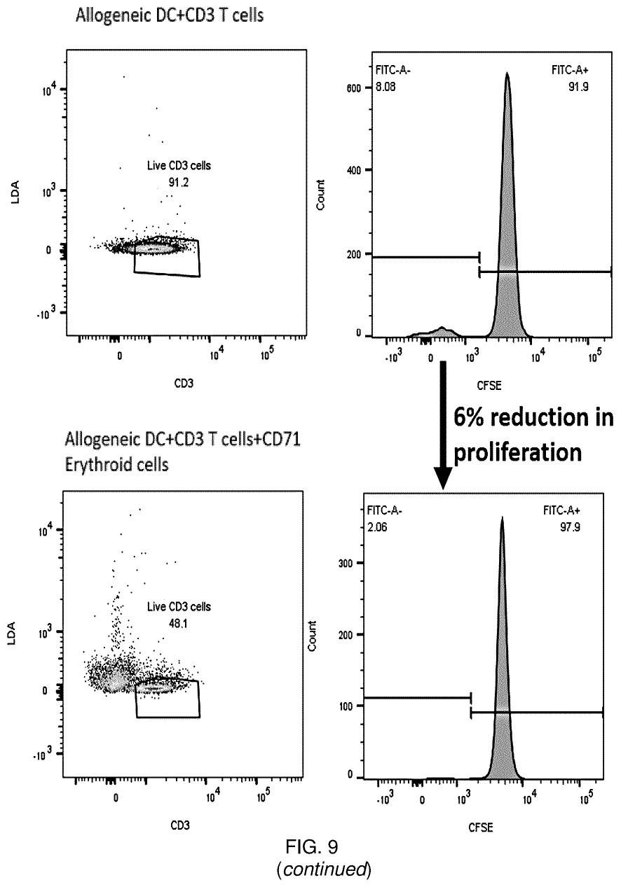

This assay uses antigen presenting cells (dendritic cells) to activate human CD3+ T cells and induce proliferation. Proliferating cells will not retain the CFSE dye and therefore a higher CFSE signal indicates less proliferative cells. Addition of the CD71+ cells to the T cell culture inhibits T cell activation and proliferation as indicated by the higher CFSE signal in the CD71+ coculture condition relative to the control condition (FIG. 9).

While several examples and embodiments of the methods are described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A method of preparing a population of CD71+ cells, comprising:
culturing pluripotent stem cells to differentiate to a fetal liver/yolk sac phenotype, such as increased expression of erythroid markers CD36, CD47, erythropoietin receptor (EPOR), and transforming growth factor β1 (TGFβ1); and
coculturing CD34⁺ hematopoietic stem and progenitor cells (HPSCs) with the differentiated pluripotent stem cells to produce an expanded population of CD71⁺ (CD71^{HI}) cells from the HPSCs.

2. The method of claim 1, wherein the pluripotent stem cells are differentiated to a fetal liver/yolk sac phenotype by expressing a gene for expressing a GATA6 protein in the pluripotent stem cells or by introducing a GATA6 protein or mRNA encoding a GATA6 protein into the pluripotent stem cells.

3. The method of claim 2, wherein the pluripotent stem cells are differentiated to a fetal liver/yolk sac phenotype by expressing a gene for expressing a GATA6 protein in the pluripotent stem cells.

4. The method of claim 2, wherein the gene for expressing a GATA6 protein is inducible in the pluripotent stem cells.

5. The method of claim 4, wherein the gene for expressing a GATA6 protein in the pluripotent stem cells is induced during the first 3 or 4 days of culture of the pluripotent stem cells.

6. (canceled)

7. The method of claim 2, comprising introducing a GATA6 protein or mRNA encoding a GATA6 protein into the pluripotent stem cells.

8. The method of claim 7, wherein the GATA6 protein or mRNA encoding a GATA6 protein is introduced into the pluripotent stem cells during the first 3 or 4 days of culture of the pluripotent stem cells.

9. The method of claim 1, wherein the pluripotent stem cells are induced pluripotent stem cells (iPSCs).

10. The method of claim 1, wherein the pluripotent stem cells are cultured on a basement membrane matrix.

11. The method of claim 1, wherein the HPSCs are obtained from cord blood or bone marrow.

12. The method of claim 1, wherein the HPSCs are obtained from a patient to whom the CD71⁺ cells are to be administered.

13. The method of claim 1, wherein the HPSCs are cocultured with the differentiated pluripotent stem cells in media comprising a factor chosen from one or more of thrombopoietin, stem cell factor, FLT3 ligand, IL-3, IL-6, and GM-CSF.

14-16. (canceled)

17. The method of claim 1, further comprising, after at least six days of coculturing the HPSCs with the differentiated pluripotent stem cells, separating cells of the expanded HPSCs comprising CD71+ cells from the differentiated pluripotent stem cells and collecting the separated cells of the expanded HPSCs comprising CD71+ cells.

18. The method of claim 17, further comprising enriching the separated cells of the expanded HPSCs comprising CD71+ cells for CD71+ cells by an affinity purification method.

19. The method of claim 18, wherein the affinity purification method is a fluorescence-activated cell sorting method, a magnetic bead separation method, a panning method, or a column purification method.

20. (canceled)

21. A cell culture device comprising: a cell culture vessel comprising cell culture media, and a coculture of cells having a fetal liver/yolk sac phenotype and CD34⁺ hematopoietic stem and progenitor cells.

22. (canceled)

23. The device of claim 21, wherein the cells having a fetal liver/yolk sac phenotype comprise an inducible gene for expressing a GATA6 protein.

24. (canceled)

25. The device of claim 21, wherein the media comprises a factor chosen from one or more of thrombopoietin, stem cell factor, FLT3 ligand, IL-3, IL-6, and GM-CSF.

26-27. (canceled)

28. The device claim 21, wherein the cells of the coculture comprises, excluding the cells having a fetal liver/yolk sac phenotype, at least 10%, at least 15%, or at least 20% CD34⁻CD71⁺ cells.

29. The device of claim 21, wherein the cells of the coculture comprises, excluding the cells having a fetal liver/yolk sac phenotype, at least 70%, at least 75%, or at least 80% CD71⁺ cells.

30. A method of immunosuppression in a patient in need thereof, having an inflammatory disease or an allogeneic or xenogeneic tissue transplant, comprising administering to the patient an immunosuppressive number of CD71⁺ or CD71^{HI} cells produced by the method of claim 1, thereby reducing a cell-mediated response in the patient.


SEQUENCE LISTING (2 sequences)

SEQ ID NO: 1 - RNA, 3624 nt, Homo sapiens
  source 1..3624: mol_type = mRNA
ggactcgcgctcgcccgctggcgccctcggcttctctccgcgcctgggagcaccctccgccgcggccgttctccatgcgcagcgcccgcccgaggagctagacgtcagcttggagcggcgccggaccgtggatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccagagttttaaaaataaaaagggtattgttttgtcttctgtacagtgagttccttcccttttcaaagctttctttttatgctgtatgtgactatagatattcatataaaacaagtgcacgtgaagtttgcaaaatgctttaaggccttcctttcaaagcatagtccttttggagccgttttgtaccttttataccttggcttatttgaagttgacacatggggttagttactactctccatgtgcattggggacagtttttataagtgggaaggactcagtattattatatttgagatgataagcattttgtttgggaacaatgcttaaaaatattccagaaagttcagattttttttctttgtgaatgaaatatattctggcccacgaacagggcgatttcctttcagttttttccttttgcaacgtgccttgaagtctcaaagctcacctgaggttgcagacgttacccccaacagaagataggtagaaatgattccagtggcctctttgtattttcttcattgttgagtagatttcaggaaatcaggaggtgtttcacaatacagaatgatggcctttaactgtg

SEQ ID NO: 2 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA